(19) United States
(12) Patent Application Publication
(10) Pub. No.: US 2023/0363982 A1
(43) Pub. Date: Nov. 16, 2023

(54) DENTAL COATING COMPOSITION

(71) Applicants: MITSUI CHEMICALS, INC., Tokyo (JP); SDC TECHNOLOGIES INC., Irvine, CA (US); KULZER GMBH, Hanau (DE)

(72) Inventors: Hideyuki ITO, Chiba (JP); Yoko KOSUGI, Chiba (JP); James Myoungsouk JANG, Irvine, CA (US); Ren-Zhi JIN, Irvine, CA (US); Sapna BLACKBURN, Mount Vernon, IN (US); Klaus RUPPERT, Maintal (DE); Andreas GRUNDLER, Usingen (DE); Andrea HAMBACH, Usingen (DE)

(21) Appl. No.: 18/245,050

(22) PCT Filed: Sep. 15, 2021
(86) PCT No.: PCT/JP2021/033914

§ 371 Date: Mar. 13, 2023

Related U.S. Application Data
(60) Provisional application No. 63/080,186, filed on Sep. 18, 2020.

(51) Int. Cl. A61K 6/20; A61K 6/62; A61K 6/71; A61C 13/09; A61C 13/00; A61C 13/15

(52) U.S. Cl. CPC A61K 6/20; A61K 6/62; A61K 6/71; A61C 13/09; A61C 13/0006; A61C 19/003

(57) ABSTRACT

Dental coating compositions that have one or more of excellent properties including plaque resistance, microbial plaque resistance, strong adhesion, excellent hardness, high wear resistance, and high color resistance are provided. Dental appliance or natural tooth covered by the dental coating compositions, and production methods of the dental coating compositions are also provided. The coating compositions include one or more of a trifunctional or higher multifunctional (meth)acrylate monomer, an inorganic filler, a photoinitiator, and an organic solvent. The coating compositions are substantially free from antibacterial chemical additives.

### TECHNICAL FIELD

Embodiments of the present disclosure relate to coating compositions for dental appliance or natural tooth. Dental coating compositions can be provided on a surface of dental appliance or natural tooth.

### BACKGROUND ART

Dental appliance, such as artificial dentures, artificial teeth, dental prostheses, and dental aligners in the oral environment are influenced by local factors including bacterial plaque, tartar, physical stress, and discoloration. Antimicrobial constituent, such as silver has been used for coating the surface of dental prosthesis. In view of dental esthetic and maintaining mechanical properties, there is a demand to decrease amount of an antimicrobial constituent used for dental appliance or natural tooth.

### SUMMARY OF INVENTION

The following summary presents a simplified summary in order to provide a basic understanding of some aspects of the devices discussed herein. This summary is not an extensive overview of the devices discussed herein. It is not intended to identify critical elements or to delineate the scope of such devices. Its sole purpose is to present some concepts in a simplified form as a prelude to the more detailed description that is presented later.

In accordance with one aspect of the present disclosure, provided is a coated dental appliance or natural tooth that includes a dental appliance or natural tooth and a coating composition affixed on at least one portion of the dental appliance or natural tooth. The coating composition includes at least one trifunctional or higher multifunctional (meth)acrylate monomer, an inorganic filler, at least one free-radical photoinitiator; and an organic solvent.

In accordance with one aspect of the present disclosure, provided is the coating composition for a dental appliance or natural tooth. The coating composition includes at least multifunctional (meth)acrylate oligomer. The multifunctional (meth)acrylate oligomer is a hexafunctional aromatic urethane (meth)acrylate oligomer. The inorganic filler is amorphous nanosilica. The trifunctional or higher functionality (meth)acrylate monomer is trimethylolpropane triacrylate. The coating composition includes difunctional (meth)acrylate monomers or oligomers. The coating composition includes 1 to 30 parts by weight of the amorphous nanosilica in the coating composition. The coating includes 5 to 25 parts by weight of amorphous nanosilica in the coating composition. The coating composition includes at least 10 weight % of an organic non-reactive solvent.

In accordance with one aspect of the present disclosure, provided is a method of producing the coated dental appliance or natural tooth that includes applying the coating composition on a surface of a dental appliance or natural tooth, drying the applied coating composition on the surface of the dental appliance or natural tooth, and curing the applied coating composition on the surface of the dental appliance or natural tooth. The dental appliance is a full denture, a partial denture, a denture base, an artificial tooth, a dental prostheses, a splint, a dental aligner, a crown or a bridge. The coating composition is applied by brushing the coating composition onto the dental appliance or natural tooth. The coating composition is applied by dipping the dental appliance into the coating composition. The coating composition is applied by spraying the coating composition onto the dental appliance or natural tooth. The drying includes air-drying at room temperature for at least 2 minutes. The coating composition is cured by light in a wavelength range of 300 nm to 500 nm. The coating composition is applied to the surface of the dental appliance or the natural tooth without any adhesion promoter or bonding agent being part of the coating composition or applied before the coating composition.

In accordance with one aspect of the present disclosure, provided is a coated dental appliance or natural tooth manufactured by the method of the present disclosure. The coated dental appliance or natural tooth reduces plaque adhesion on its surface when compared to a corresponding uncoated dental appliance or natural tooth by more than 50% after 23 hours of incubation. The coated dental appliance or natural tooth reduces plaque adhesion on its surface when compared to a corresponding uncoated dental appliance or natural tooth by more than 70% after 23 hours of incubation. The coated dental appliance or natural tooth reduces plaque adhesion on its surface when compared to a corresponding uncoated dental appliance or natural tooth by more than 80% after 23 hours of incubation. The coated dental appliance or natural tooth has a surface hardness of at least 9 N as measured by Erichsen Pencil Hardness test. The cured coating composition on the dental appliance or natural tooth has a layer thickness of 1 to 100 micrometre. The cured coating composition on the dental appliance or natural tooth has a layer thickness of 3 to 60 micrometre. The cured coating composition is optically clear and colorless.

In accordance with one aspect of the present disclosure, provided is a coating composition for a dental appliance or natural tooth, including a (meth)acrylate component including a trifunctional or higher multifunctional (meth)acrylate monomer, an inorganic filler, a photoinitiator, and an organic solvent. A water contact angle of a cured coating of the coating composition is 75 degrees or more and less than 90 degrees. A water contact angle of a cured coating of the coating composition is 81 degrees or more. Surface roughness of a cured coating of the coating composition after 10,000 cycles of tooth brushing is 0.100 μm or less. Coffee discoloration resistance of a cured coating of the coating composition is 3.00 ΔE*ab or less. The coating composition further includes 2-hydroxy-2-methyl-1-phenyl-propan-1-one and 1-hydroxy-cyclohexyl-phenyl-ketone. The (meth)acrylate component further includes a multifunctional urethane (meth)acrylate oligomer. The (meth)acrylate component includes 80 weight % or more and 100 weight % or less of the trifunctional or higher multifunctional (meth)acrylate monomer and the multifunctional urethane (meth)acrylate oligomer with respect to a total weight of the (meth)acrylate component.

### BEST MODE FOR CARRYING OUT THE INVENTION

A present disclosure provides dental coating compositions that are anti-plaque and abrasion-resistant coatings for dental appliance or natural tooth. The dental coating compositions of the present disclosure have one or more of excellent properties including microbial resistance, strong adhesion, excellent hardness, high wear resistance, and high color resistance. A present disclosure further provides dental appliance or natural tooth covered by the dental coating compositions, and production methods of the dental coating compositions.

In accordance with the embodiments of the present disclosure, the coating composition includes a trifunctional or higher multifunctional (meth)acrylate monomer; an inorganic filler; a photoinitiator; and an organic solvent.

In one embodiment, the coating composition is substantially free from antibacterial chemical additives. In other words, no antibacterial chemical additive is added to the coating compositions of the present disclosure. In one embodiment, the coating composition includes less than 10 weight % of antibacterial chemical additives, preferably less than 1 weight % of antibacterial chemical additives, more preferably less than 0.1 weight % of antibacterial chemical additives. Examples of the antibacterial chemical additives include silver, zinc, copper, chlorhexidine, cethylpyridinium chloride, benzalkonium chloride and antibiotics.

### Multifunctional (Meth)Acrylate Component

The coating compositions disclosed herein include a (meth)acrylate component including one or more of multifunctional (meth)acrylate monomers. The coating compositions can also include a (meth)acrylate component including one or more multifunctional (meth)acrylate oligomers. Oligomers are formed when a few number of monomers are linked together via covalent bonds. In accordance with certain embodiments of the coating compositions disclosed herein, examples of the multifunctional (meth)acrylate oligomer of the present disclosure include 2-100 numbers of the multifunctional (meth)acrylate monomers.

The multifunctional (meth)acrylate monomers have at least two or more acrylate functionalities. (Meth)acrylate functionality is defined herein as the number of functional groups represented by the formula CH2=CRC═OO, where R is a hydrogen or a methyl group, that are present in one (1) mole of the component, i.e., one (1) mole of the multifunctional (meth)acrylate monomer component or one (1) mole of the multifunctional (meth)acrylate oligomer component. As an example of (meth)acrylate functionality, a trimethylolpropane triacrylate monomer is represented by the formula (CH2=CHC═OOCH₂)₃CC₂H₅. As shown in its formula, trimethylolpropane triacrylate contains three (3) functional groups represented by the formula CH₂═CRC═OO per one (1) mole of the monomer, where R is a hydrogen (H). Therefore, as defined herein, the trimethylolpropane triacrylate monomer is a (meth)acrylate monomer that has a (meth)acrylate functionality of three (3). In another example, a neopentyl glycol dimethacrylate monomer is represented by the formula [CH₂═C(CH₃)C═OOCH₂]₂C(CH₃)₂. As shown in its formula, neopentyl glycol dimethacrylate contains two (2) functional groups represented by the formula CH₂═CRC═OO per one (1) mole of monomer, where R is a methyl group (CH₃). Therefore, as defined herein, the neopentyl glycol dimethacrylate monomer is a (meth)acrylate monomer that has a (meth)acrylate functionality of two (2).

Thus, in accordance with certain embodiments of the coating compositions disclosed herein, if a single type of (meth)acrylate monomer is present in the multifunctional (meth)acrylate monomer component, the single type of (meth)acrylate monomer present has a (meth)acrylate functionality of about two (2) or greater. In view of increasing plaque resistance, the coating compositions disclosed herein include a trifunctional or higher multifunctional (meth)acrylate monomer. Examples of suitable types of (meth)acrylate monomers that can be used alone in the multifunctional (meth)acrylate monomer component include, but are not limited to, triacrylates, tetraacrylates, pentaacrylates, hexaacrylates, and the like. In accordance with certain embodiments of the coating compositions disclosed herein, the coating compositions can also include diacrylates.

Examples of suitable diacrylate monomers that are used as the multifunctional (meth)acrylate monomer component in accordance with this embodiment include, but are not limited to, 1,6-hexanediol diacrylate; ethylene glycol diacrylate; ethylene glycol dimethacrylate; diethylene glycol diacrylate; triethylene glycol diacrylate; triethylene glycol dimethacrylate; tetraethylene glycol diacrylate; tetraethylene glycol dimethacrylate; polyethylene glycol diacrylate; tripropylene glycol diacrylate; triisopropylene glycol diacrylate; polypropylene glycol dimethacrylate; 1,4-butanediol diacrylate; 1,4-butanediol dimethacrylate; poly(butanediol) diacrylate; neopentyl glycol diacrylate; neopentyl glycol dimethacrylate; 1,3-butylene glycol diacrylate; 1,3-butylene glycol dimethacrylate; 1,12-dodecanediol dimethacrylate; alkoxylated aliphatic diacrylates such as alkoxylated hexanediol diacrylates and alkoxylated neopentyl glycol diacrylates, e.g., propoxylated neopentyl glycol diacrylate; cyclic or polycyclic diacrylates such as cyclohexane dimethanol diacrylate and tricyclodecane dimethanol diacrylate; bisphenol-A diacrylate; bisphenol-A dimethacrylate; alkoxylated bisphenol-A diacrylates such as ethoxylated bisphenol-A diacrylate; alkoxylated bisphenol-A dimethacrylates such as ethoxylated bisphenol-A dimethacrylate; and polyester diacrylates.

Examples of suitable triacrylate monomers that are used as the multifunctional (meth)acrylate monomer component in accordance with this embodiment include, but are not limited to, trimethylolpropane triacrylate, trimethylolpropane trimethacrylate, pentaerythritol monohydroxy triacrylate, ethoxylated trimethylolpropane triacrylate, propoxylated glyceryl triacrylate, propoxylated trimethylolpropane triacrylate, and tris(2-hydroxyethyl) isocyanurate triacrylate.

Examples of suitable tetraacrylate monomers that are used as the multifunctional (meth)acrylate monomer component in accordance with this embodiment include, but are not limited to, pentaerythritol tetraacrylate and di-trimethylolpropane tetraacrylate.

Examples of suitable pentaacrylate monomers that are used as the multifunctional (meth)acrylate monomer component in accordance with this embodiment include, but are not limited to, dipentaerythritol pentaacrylate and sorbitol pentaacrylate.

An example of a suitable hexaacrylate monomer that is used as the multifunctional (meth)acrylate monomer component in accordance with this embodiment includes, but is not limited to, dipentaerythritol hexaacrylate.

In other embodiments, the multifunctional (meth)acrylate monomer component includes more than one type of (meth)acrylate monomer. In accordance with this embodiment, the multifunctional (meth)acrylate monomer component can include individual (meth)acrylate monomers that have a (meth)acrylate functionality of at least two or more.

Examples of suitable types of (meth)acrylate monomers that can be used in accordance with this embodiment, i.e., the embodiment that has more than one type of (meth)acrylate monomer in the multifunctional (meth)acrylate monomer component, include, but are not limited to, monoacrylates, diacrylates, triacrylates, tetraacrylates, pentaacrylates, hexaacrylates, and the like. The examples of diacrylates, triacrylates, tetraacrylates, pentaacrylates, and hexaacrylates disclosed above are suitable for use as some or all of the (meth)acrylate monomers in the multifunctional (meth)acrylate monomer component in accordance with this embodiment.

In other embodiments, the preferred multifunctional (meth)acrylate monomer component is aliphatic trifunctional or higher functionality (meth)acrylate component, and more preferably trimethylolpropane triacrylate.

In accordance with all embodiments of the coating compositions described herein, the multifunctional (meth)acrylate monomer component has (meth)acrylate functionality ranging from greater or equal to 2 to about 10, preferably ranging from about 2 to about 6.

The content of the (meth)acrylate component in the coating composition is preferably from 1% by mass to 30% by mass, more preferably from 3% by mass to 20% by mass, and still more preferably 5% by mass to 15% by mass, with respect to the total amount of the coating composition. The content of the multifunctional (meth)acrylate monomer component (the total content, in a case in which two or more types thereof are included) in the coating composition is preferably from 1% by mass to 30% by mass, more preferably from 3% by mass to 20% by mass, and still more preferably 5% by mass to 15% by mass, with respect to the total amount of the coating composition. Especially, the content of trifunctional or higher functionality (meth)acrylate component is preferably from 1% by mass to 20% by mass, more preferably from 3% by mass to 15% by mass, and still more preferably 5% by mass to 10% by mass, with respect to the total amount of the coating composition.

In one embodiment, the (meth)acrylate component further includes a multifunctional urethane (meth)acrylate oligomer. The (meth)acrylate component includes 80 weight % or more and 100 weight % or less of the trifunctional or higher multifunctional (meth)acrylate monomer and the multifunctional urethane (meth)acrylate oligomer with respect to a total weight of the (meth)acrylate component. In one embodiment, (meth)acrylate component further includes a multifunctional urethane (meth)acrylate oligomer. The (meth)acrylate component includes 85 weight % or more and 100 weight % or less of the trifunctional or higher multifunctional (meth)acrylate monomer and the multifunctional urethane (meth)acrylate oligomer with respect to a total weight of the (meth)acrylate component. In one embodiment, the (meth)acrylate component further includes a multifunctional urethane (meth)acrylate oligomer. The (meth)acrylate component includes 90 weight % or more and 100 weight % or less of the trifunctional or higher multifunctional (meth)acrylate monomer and the multifunctional urethane (meth)acrylate oligomer with respect to a total weight of the (meth)acrylate component. In one embodiment, the (meth)acrylate component further includes a multifunctional urethane (meth)acrylate oligomer. The (meth)acrylate component includes 95 weight % or more and 100 weight % or less of the trifunctional or higher multifunctional (meth)acrylate monomer and the multifunctional urethane (meth)acrylate oligomer with respect to a total weight of the (meth)acrylate component.

In accordance with one or more embodiments, weight-average molecular weight (Mw) of the multifunctional (meth)acrylate monomer in the coating compositions disclosed herein is preferably 150 to 1000, more preferably 200 to 500.

### Multifunctional (Meth)Acrylate Oligomer Component

The coating composition disclosed herein can include a multifunctional (meth)acrylate oligomer component. The multifunctional acrylate oligomer component comprises one or more (meth)acrylate oligomers that collectively have at least two or more acrylate functionalities, in accordance with the definition of (meth)acrylate functionality previously disclosed herein.

In accordance with preferred embodiments of the coating compositions disclosed herein, a multifunctional (meth)acrylate oligomer component disclosed herein includes a multifunctional urethane (meth)acrylate oligomer component. The multifunctional urethane acrylate oligomer component comprises one or more urethane (meth)acrylate oligomers that collectively have at least two or more acrylate functionalities, in accordance with the definition of (meth)acrylate functionality previously disclosed herein. Each of the one or more urethane (meth)acrylate oligomers that collectively form the multifunctional urethane (meth)acrylate oligomer component are characterized by the occurrence of one or more urethane groups represented by the formula NHC═OO. Such urethane (meth)acrylate oligomers are prepared by reacting multifunctional isocyanates with polyols to form monomers having the one or more urethane groups. The monomers having one or more urethane groups are then end-capped with (meth)acrylate monomers by reacting the monomers having one or more urethane groups with hydroxyl-functional (meth)acrylate monomers, thereby resulting in a urethane (meth)acrylate oligomer. One of ordinary skill in the art would understand how to control the (meth)acrylate functionality of the urethane (meth)acrylate oligomer to form multifunctional oligomers suitable for the multifunctional urethane (meth)acrylate oligomer component.

In accordance with certain embodiments of the coating compositions disclosed herein, if a single type of urethane (meth)acrylate oligomer is present in the multifunctional urethane (meth)acrylate oligomer component, the single type of urethane (meth)acrylate oligomer present has a (meth)acrylate functionality of about two (2) or greater. Examples of suitable types of urethane (meth)acrylate oligomers that can be used alone in the multifunctional urethane (meth)acrylate oligomer component include, but are not limited to, urethane diacrylates, urethane triacrylates, urethane tetraacrylates, urethane pentaacrylates, urethane hexaacrylates, and the like. Preferably, in accordance with this and other embodiments, the urethane (meth)acrylate oligomers used alone in the multifunctional urethane (meth)acrylate oligomer component are aliphatic urethane (meth)acrylate oligomers.

In other embodiments, the multifunctional urethane (meth)acrylate oligomer component can include more than one type of urethane (meth)acrylate oligomer. In accordance with this embodiment, the multifunctional (meth)acrylate monomer component can include individual urethane (meth)acrylate oligomers that have at least two or more (meth)acrylate functionality.

Examples of suitable urethane (meth)acrylate oligomers that can be used in accordance with this embodiment, i.e., the embodiment that has more than one type of urethane (meth)acrylate oligomers in the multifunctional urethane (meth)acrylate oligomer component, include, but are not limited to, urethane diacrylates, urethane triacrylates, urethane tetraacrylates, urethane pentaacrylates, urethane hexaacrylates, and the like. Preferably, in accordance with this and other embodiments, the urethane (meth)acrylate oligomers used in combination with other urethane (meth)acrylate oligomers in the multifunctional urethane (meth)acrylate oligomer component are aliphatic urethane (meth)acrylate oligomers. In accordance with certain embodiments, aromatic urethane (meth)acrylate oligomers are used in combination with aliphatic urethane (meth)acrylate oligomers as the multifunctional urethane (meth)acrylate oligomer component in the coating compositions disclosed herein.

In accordance with all embodiments of the coating compositions described herein, the multifunctional urethane (meth)acrylate oligomer component has an average (meth)acrylate functionality ranging from greater than 1 to about 10, preferably ranging from about 2 to about 6, and more preferably ranging from about 4 to about 6.

Examples of suitable urethane (meth)acrylate oligomers that can be used in accordance with one embodiment of the present disclosure include hexafunctional aromatic urethane (meth)acrylate oligomer, or include hexafunctional aliphatic urethane (meth)acrylate oligomer and hexafunctional aromatic urethane (meth)acrylate oligomer.

The content of the multifunctional urethane (meth)acrylate oligomer component (the total content, in a case in which two or more types thereof are included) in the coating composition is preferably from 5% by mass to 65% by mass, more preferably from 10% by mass to 55% by mass, and still more preferably 20% by mass to 50% by mass, with respect to the total amount of the coating composition. Especially, the content of hexafunctional aromatic urethane (meth)acrylate oligomer is preferably from 5% by mass to 65% by mass, more preferably from 10% by mass to 55% by mass, and still more preferably 20% by mass to 50% by mass, with respect to the total amount of the coating composition.

### Inorganic Filler

The coating compositions disclosed herein include an inorganic filler. Example inorganic fillers of the present disclosure include a colloidal silica component, e.g., colloidal nanosilica. The colloidal silica component comprises one or more types of colloidal silica. Colloidal silica, as used herein, refers to a dispersion of fine, amorphous particles of silica, i.e., SiO2, in a liquid phase. Suitable liquid phases for the colloidal silica component include water, an organic solvent, or combinations of water and an organic solvent. Generally, the silica particles in a colloidal dispersion are spherical in nature. In accordance with embodiments disclosed herein, the one or more colloidal silica used as the colloidal silica component have a solids content ranging from about 20% to about 70% by weight, preferably from about 30% to about 60% by weight, and have a mean particle diameter size up to about 200 nm, preferably less than about 100 nm, and more preferably less than about 50 nm. Colloidal silica is generally available in acidic or basic form, either of which are used in the coating compositions disclosed herein. In an embodiment, the colloidal silica used with the coating compositions disclosed herein is 40% colloidal nanosilica, for example, in 1-methoxyl-2-propanol, 60% colloidal nanosilica, for example, in 1-methoxyl-2-propanol, 30% colloidal nanosilica, for example, in 2-propoxyethanol, and 30% colloidal nanosilica, for example, in water.

The colloidal silica, when added to the coating compositions disclosed herein, is considered a reactive material. This is due, at least in part, to hydroxyl functional groups present on the surface of the colloidal silica particles. These surface bound hydroxyls can react with other components in the coating composition. Therefore, the order in which the colloidal silica is added during the preparation of the coating compositions disclosed herein can be determinative of the final properties of the cured coating composition because the colloidal silica may react differently with different components at different points during its preparation, thereby providing different properties in the cured coating. The colloidal silica component can be added during the preparation of the coating compositions disclosed herein after the acid is added but before the multifunctional urethane (meth)acrylate component is added. In accordance with one or more embodiments, the colloidal silica can be added during the preparation of the coating compositions disclosed herein under vigorous agitation, and left to stir for a period of time, for about 0.50 to about 1.50 hours, before the multifunctional urethane (meth)acrylate component is added.

The addition of the colloidal silica to the coating compositions enhances the abrasion resistance of the cured coating. However, the addition of too much of the colloidal silica negatively impacts film formation for the cured coating. In accordance with the embodiments disclosed herein, the coating compositions disclosed herein include up to about 35 weight % colloidal silica by weight of solids of the coating composition.

The content of the inorganic filler (the total content, in a case in which two or more types thereof are included, preferably, the content of the colloidal silica) in the coating composition is preferably from 1% by mass to 30% by mass, more preferably from 5% by mass to 28% by mass, and still more preferably 10% by mass to 25% by mass, with respect to the total amount of the coating composition.

### Photoinitiator

In accordance with one or more embodiments, the coating compositions disclosed herein include a photoinitiator (e.g., free-radical photoinitiators). The photoinitiator present in the coating composition initiates and advances the crosslinking, i.e., curing, of the coating composition when the coating composition is exposed to any suitable light in the wavelength range of 300 to 500 nm. Example light sources include tungsten halogen, light-emitting diodes (LED), plasma arcs, and lasers. The photoinitiators do this by generating radicals when exposed to the light. The radicals, in turn, initiate and advance the polymerization, i.e., crosslinking, of the coating composition during cure. The photoinitiator can be added at any point during the process for preparing the coating composition.

Examples of suitable light sensitive photoinitiators or blends of initiators used in coating compositions disclosed herein include, but are not limited to, benzoin; substituted benzoins such as butyl isomers of benzoin ethers; benzophenone; substituted benzophenones such as hydroxy benzophenone; 2-hydroxyethyl-N-maleimide; 2-[2-hydroxyethyl(methyl)amino]ethanol anthraquinone; thioxanthone; α,α-diethoxyacetophenone; 2,2-dimethoxy-1,2-diphenylethan-1-one; 2-hydroxy-2-methyl-1-phenyl-propan-1-one; diphenyl(2,4,6-trimethylbenzoyl)phosphine oxide, phenyl glyoxylic acid methyl ester; 1-hydroxylcyclohexyl phenyl ketone; 2-benzyl-2-dimethylamino-1-(4-morpholinophenyl)-butanone-1; 2-dimethylamino-2-(4-methyl-benzyl)-1-(4-morpholin-4-yl-phenyl)-butan-1-o-ne; 2-methyl-1-[4-(methylthio)phenyl]-2-morpholinopropan-1-one; 1-[4-(2-hydroxyethoxy)-phenyl]-2-hydroxy-2-methyl-1-propane-1-one, 1-hydroxy-cyclohexyl-phenyl-ketone, and 2-hydroxy-2-methyl-1-phenyl-propan-1-one.

In other embodiments, in order to achieve sufficient cure of the coating composition, preferably the photoinitiator comprises both 2-hydroxy-2-methyl-1-phenyl-propan-1-one and 1-hydroxy-cyclohexyl-phenyl-ketone.

In accordance with one or more embodiments, the coating compositions disclosed herein are optionally cured using electron beam (EB) radiation. The coating compositions in accordance with this embodiment do not require a photoinitiator. However, because EB radiation has a wavelength that is different from 300 to 500 nm, the coating cured using EB radiation may have a different crosslink density as compared to the coating cured using the light in wavelength of 300 to 500 nm, all other conditions being held equal. One of ordinary skill in the art would recognize this and adjust the cure parameters to obtain the antimicrobial and abrasion-resistant coatings obtained using EB radiation.

The content of the photoinitiator (the total content, in a case in which two or more types thereof are included) in the coating composition is preferably from 0.1% by mass to 10% by mass, more preferably from 0.5% by mass to 8% by mass, and still more preferably 1% by mass to 5% by mass, with respect to the total amount of the coating composition.

### Organic Solvent Component

The coating compositions disclosed herein include an organic solvent component. The organic solvent component comprises one or more organic solvents that are added during the preparation of the coating composition. These one or more organic solvents may be added at different points during the preparation of the coating composition. These one or more organic solvents are added as an individual solvent component or as part of another component. For example, other components used in the coating compositions disclosed herein, such as the colloidal silica component or the multifunctional urethane (meth)acrylate oligomer component, may be in a solution, suspension, or dispersion form and consequently contain amounts of organic solvent that are added along with its respective component (e.g., the colloidal silica or the multifunctional urethane (meth)acrylate oligomer) to the coating composition. In such embodiments, any residual organic solvent present with other components used in the coating composition is considered to be part of the one or more of the organic solvents that collectively form the organic solvent component of the coating composition.

In accordance with one or more embodiments of the coating compositions disclosed herein, during the process for preparing the coating composition, any one component of the coating composition can be mixed with an organic solvent component comprising a first organic solvent to form a solution. One or more additional organic solvents, e.g., a second organic solvent, a third organic solvent, etc., which may be the same or different as the first organic solvent, can be added at different points during the process for preparing of the coating composition. As discussed above, the one or more additional organic solvents added at different points can be part of another component, e.g., the colloidal silica component, the multifunctional urethane (meth)acrylate oligomer component, etc.

Examples of suitable organic solvents that are used as the organic solvent component in the coating compositions disclosed herein include, but are not limited to, ketones such as methylethylketone, methylisobutyl ketone, diacetone alcohol, 3,3-dimethyl-2-butanone, pentanedione, and the like; esters such as n-butyl acetate, isobutyl acetate, and propylene glycol methyl ether acetate; the glycol ethers disclosed herein; propanediol, such as 1-methoxy-2-propanol, 2-propoxyethanol, and alcohols such as the primary and secondary alcohols (e.g., isopropyl alcohol) disclosed herein as well as the ether alcohols disclosed herein.

The content of the organic solvent component (the total content, in a case in which two or more types thereof are included) in the coating composition is preferably from 5% by mass to 90% by mass, more preferably from 7% by mass to 80% by mass, and still more preferably 10% by mass to 70% by mass, with respect to the total amount of the coating composition.

### Amino-Organofunctional Silane

The coating compositions disclosed herein can include an adduct of a multifunctional (meth)acrylate monomer/oligomer component and an amino-organofunctional silane. The adduct is formed by treating the multifunctional (meth)acrylate monomer/oligomer with suitable amino-organofunctional silanes. Suitable amino-organofunctional silanes used to form the adduct are represented by the following formula:

XₐSi[Q¹(NHQ²)_{b}NZH]₄₋ₐ, (I)

where X is an alkoxy group having from 1 to 6 carbon atoms; Q¹ and Q² are the same or different divalent hydrocarbon groups; Z is a hydrogen or a monovalent hydrocarbon group; a is an integer from 1 to 3; and b is an integer from 0 to 6.

In accordance with one embodiment of the coating compositions disclosed herein, Q¹ and Q² in formula (I) are the same or different divalent hydrocarbon groups that are represented by the formula (CH₂)ₙ, where n is an integer from 1 to 10, preferably from 1 to 6. In accordance with this or other embodiments, Z in formula (I) is a monovalent hydrocarbon group comprising an alkyl radical containing 1 to 10 carbon atoms, preferably from 1 to 6 carbon atoms.

Examples of amino-organofunctional silanes represented by formula (I) above include 3-aminopropyltrimethoxysilane, 3-aminopropyltriethoxysilane (gamma-aminopropyltriethoxysilane), n-(2-aminoethyl)-3-aminopropyltrimethoxysilane, and 3-(phenylamino)propyltrimethoxysilane.

### Adduct

The adduct that can be used in the coating compositions disclosed herein is the reaction product of the multifunctional (meth)acrylate monomer/oligomer component disclosed herein and the amino-organofunctional silane disclosed herein. In accordance with one or more embodiments, the adduct used in the coating composition is the reaction product of a Michael reaction where the amino functionality of the amino-organofunctional silane is the Michael donor and the (meth)acrylate functionality (specifically, the α,β unsaturated carbonyl) of the multifunctional (meth)acrylate monomer/oligomer component is the Michael acceptor. In the coating compositions disclosed herein, the molar ratio of the average (meth)acrylate functionality of the multifunctional (meth)acrylate monomer/oligomer component to the amino functionality of the amino-organofunctional silane reacted to form the adduct is greater than one (1), preferably about two (2) or greater.

The Michael reaction takes place using neat form reactants, i.e., a neat form multifunctional (meth)acrylate monomer/oligomer component and a neat form amino-organofunctional silane. The term “neat form” used herein refers to a purity of 100% by weight of the particular reactant. Alternatively, the Michael reaction takes place in solution. In certain embodiments, one reactant, i.e., the multifunctional (meth)acrylate monomer/oligomer component or amino-organofunctional silane, is in neat form and the other reactant is in solution. With respect to a solution-type Michael reaction or the combined neat form and solution-type reaction, suitable solvents comprise polar organic solvents including primary or secondary alcohols such as methanol, ethanol, n-propanol, isopropanol, n-butanol, isobutanol, and the like. Other examples of suitable polar organic solvents include ether alcohols such as ethoxyethanol, butoxyethanol, methoxypropanol, and the like. Examples of other suitable solvents include glycol ethers such as propylene glycol methyl ether (PM glycol ether), dipropylene glycol methyl ether, ethylene glycol n-butyl ether, diethylene glycol n-butyl ether, and the like. In accordance with one or more embodiments, the preferred solvent used to prepare the adduct is either isopropanol or 1-methoxy-2-propanol.

The time needed to form the adduct depends on various factors, such as the temperature and solvent of the reaction. It could take about 2 hours to about 72 hours to sufficiently react the multifunctional (meth)acrylate monomer/oligomer component and the amino-organofunctional silane, whether reacted in solution, neat form, or combined solution and neat form, to prepare the adduct suitable for use in the coating compositions disclosed herein.

In one embodiment of the present disclosure, the coating compositions disclosed herein do not include an adduct of a multifunctional (meth)acrylate monomer/oligomer component and an amino-organofunctional silane. In one embodiment, the multifunctional (meth)acrylate monomer/oligomer component is not treated by silane.

When a multifunctional (meth)acrylate monomer/oligomer component is treated by silane, it is believed that the composition becomes less stable. When coating compositions include multifunctional (meth)acrylate monomer/oligomer components that are not treated by silane (e.g., gamma-aminopropyltriethoxysilane), the coating compositions exhibit higher stability and longer shelf life.

### Leveling Agents

In accordance with one or more embodiments, the coating compositions disclosed herein include a leveling agent. The leveling agent, which may also be known as a flow-control agent, is incorporated into the coating compositions described herein to spread the composition more evenly or level on the surface of the dental appliance or natural tooth and to provide substantially uniform contact with the dental appliance or natural tooth. The amount of the leveling agent can vary widely but preferably is used in an amount ranging from about 0.01 weight % to about 10 weight % leveling agent in the coating composition. Any conventional, commercially available leveling agent which is compatible with the coating composition, which is capable of leveling the coating composition on a dental appliance or natural tooth, and which enhances wetting between the coating composition and the dental appliance or natural tooth is employed. Non-limiting examples of such leveling agents include polyethers, silicones, fluorosurfactants, polyacrylates, silicone polyacrylates such as silicone hexaacrylate, and fluoro-modified polyacrylates.

### Additives

The coating compositions of the present disclosure can include other additives. Examples of the other additives include UV light stabilizers as described in Calbo, Leonard J., “The Handbook of Coatings Additives,” 2nd Ed. (Marcel Dekker, Inc., 2004), pp. 159-234, the contents of which are incorporated by reference herein. In the embodiments of the coating composition disclosed herein that are cured using UV radiation, one of ordinary skill in the art would recognize the type and amount of UV light stabilizers that may be used to impart increased weatherability to the coating compositions of the present disclosure.

### Reduction of Plaque Adhesion

In accordance with one or more embodiments, the coated dental appliance or natural tooth disclosed herein preferably reduces plaque adhesion on its surface when compared to a corresponding uncoated dental appliance or natural tooth by more than 50% after 23 h of incubation, more preferably more than 70%, more preferably more than 80%. A method of measuring reduction of plaque adhesion of the coated dental appliance or natural tooth in the present disclosure is described in the section of “Plaque Adhesion to Coated Material” in details.

### Water Contact Angle

In accordance with one or more embodiments, a water contact angle of the cured coating of the coating composition disclosed herein is preferably 75 degrees or more, more preferably 81 degrees or more, in view of increasing microbial resistance of the cured coating. In view of increasing stability, a water contact angle of the cured coating of the coating composition disclosed herein is preferably 90 degrees or less, more preferably 88 degrees or less. A water contact angle of the cured coating which is obtained by light irradiation to the coating composition is measured using a water contact angle meter.

Surface roughness after Toothbrush Abrasion

In accordance with one or more embodiments, surface roughness of the cured coating of the coating composition disclosed herein after 10,000 cycles of tooth brushing is preferably 0.500 μm or less, more preferably 0.100 μm or less. In accordance with one or more embodiments, surface roughness of the cured coating of the coating composition disclosed herein after 10,000 cycles of tooth brushing may be 0.050 μm or more. A method of measuring surface roughness of the cured coating of the coating composition after 10,000 cycles of tooth brushing in the present disclosure is described in the section of “Toothbrush Abrasion Test” in details.

### Coffee Discoloration Resistance

In accordance with one or more embodiments, coffee discoloration resistance of the cured coating of the coating composition disclosed herein is preferably 3.00 ΔE*ab or less, more preferably 1.50 E*ab or less, more preferably 1.00 ΔE*ab or less, more preferably 0.50 ΔE*ab or less. In accordance with one or more embodiments, coffee discoloration resistance of the cured coating of the coating composition disclosed herein may be 0.30 ΔE*ab or more. Coffee discoloration of the cured coating is measured using integrating sphere spectrophotometer after soaked and maintained in the coffee solution at 37 degree C. for 4 weeks. A method of measuring coffee discoloration of the cured coating of the coating in the present disclosure is described in the section of “Color Resistance of Coated Material” in details.

The coating compositions disclosed herein are applied as a coating to dental appliance or natural tooth, or to firm dental appliance surfaces or natural tooth surfaces. Example dental appliance includes a full denture, a partial denture, a denture base, an artificial tooth, a dental prostheses, a splint, a dental aligner, a crown or bridge. Example materials of the dental appliance include ceramics, titanium and its alloys, and zirconia.

The coating compositions described herein are applied in any suitable manner to the dental appliance. For example, the compositions of the present disclosure are applied to a solid dental appliance by conventional methods, such as brushing, spray coating, dip coating, and the like to form a continuous surface film on the dental appliance. In one embodiment, the coating compositions are then air-dried and cured by exposing the coated dental appliance to light in the wavelength range of 300 to 500 nm. Example light sources include tungsten halogen, light-emitting diodes (LED), plasma arcs, and lasers.

In accordance with one or more embodiments, the coating is preferably applied to the surface of the dental appliance or the natural tooth without any adhesion promoter or bonding agent being part of the coating composition or applied before the coating composition.

The covered dental appliance has a layer of the coating compositions cured on the surface of the dental appliance. In one embodiment, a thickness of the layer is 1 to 100 micrometre. In another embodiment, a thickness of the layer is 3 to 60 micrometre. In still another embodiment, the cured coating layer is optically clear and colorless.

As used in the description of the present disclosure and the appended claims, the singular forms “a,” “an,” and “the” are intended to include the plural forms as well, unless the context clearly indicates otherwise. All references incorporated herein by reference are incorporated in their entirety unless otherwise stated. The following examples are for purposes of illustration only and are not intended to limit the scope of the claims which are appended hereto.

### Materials Used

The present disclosure uses abbreviations of name of materials as shown in Table 1. Details of the materials are also shown in Table 1.

TABLE 1

| Abbreviation | Trade/Chemical Name | Supplier | Description |
|---|---|---|---|
| EB5129 | EBECRYL 5129 | Allnex, | Hexafunctional aliphatic |
| | | Alpharetta, GA | urethane acrylate oligomer |
| EB9113 | EBECRYL 9113 | Allnex, | Hexafunctional aromatic |
| | | Alpharetta, GA | urethane acrylate oligomer in |
| | | | 50% n-Butyl Acetate |
| SR238 | SR 238 | Arkema Inc., | Difunctional acrylate |
| | (1,6-Hexanediol | King of Prussia, PA | monomer |
| | Diacrylate) | | |
| SR351 | SR 351 | Arkema Inc., | Trifunctional acrylate |
| | (Trimethylolpropane | King of Prussia, PA | monomer |
| | triacrylate) | | |
| Irga 1173 | IRGACURE 1173 | BASF, | Photoinitiator |
| | (2-Hydroxy-2-methyl-1- | Florham Park, NJ | |
| | phenyl-propan-1-one) | | |
| Irga 184 | IRGACURE 184 | BASF, | Photoinitiator |
| | (1-Hydroxy-cyclohexyl- | Florham Park, NJ | |
| | phenyl-ketone) | | |
| EB1360 | EBECRYL 1360 | Allnex, | Surfactant |
| | | Alpharetta, GA | |
| M003 | M003 | SDC Technologies, Inc., | 40% Colloidal nanosilica in |
| | | Irvine, CA | PM |
| A1100 | SILQUEST A-1100 | Momentive | Amino-functional silane |
| | (Gamma-aminopropyl | Performance Materials, | coupling agent |
| | triethoxysilane) | Friendly, WV | |
| Acrylic Acid | ACRYLIC ACID | Sigma-Aldrich, | Acrylic acid |
| | | Saint Louis, MO | |
| ST30 | NPC-ST-30 | Nissan Chemical | 30% Colloidal nanosilica |
| | | America Corporation, | in 2-propoxyethanol |
| | | Houston, TX | |
| 1034A | NALCO 1034A | Nalco Company, | 30% Colloidal nanosilica in |
| | | Naperville, IL | water |
| PM | 1-Methoxy-2-Propanol | Univar, | Organic solvent |
| | | Downers Grove, IL | |
| IPA | Isopropyl Alcohol | KMG Electronic | Organic solvent |
| | | Chemicals, Inc., Fort | |
| | | Worth, TX | |
| SIMPLE GREEN | SIMPLE GREEN 13005 | Sunshine Makers, Inc., | Aqueous detergent solution |
| | | Huntington Harbour, CA | |

### Comparative Example 1

A mixture of 10.0 g of EB5129, 27.2 g of M003, and 28.3 g of PM was stirred at room temperature in a plastic beaker for 0.5 hour. Then 32 g of EB9113 was added to the mixture and stirred for another 0.5 hour, followed by an addition of 0.9 g of IRGA 1173, 1.5 g of IRGA 184, and 0.1 g of EB1360. The resulting mixture was stirred at room temperature for 4 hours and then filtered through a 1.2 micrometre filter.

### Example 1

A mixture of 5.0 g of SR351, 2.0 g of SR238, 27.3 g of M003, and 16.4 g of PM was stirred at room temperature in a plastic beaker for 0.5 hour. Then 46.8 g of EB9113 was added to the mixture and stirred for another 0.5 hour, followed by an addition of 0.9 g of IRGA 1173, 1.5 g of IRGA 184, and 0.1 g of EB1360. The resulting mixture was stirred at room temperature for 4 hours and then filtered through a 1.2 micrometre filter.

### Example 2

A mixture of 9.2 g of SR351, 3.5 g of SR238, and 1.7 g of A1100 was stirred at room temperature in a plastic beaker for 3 hours. Then 0.6 g of Acrylic Acid was added to the mixture and stirred for another 0.5 hour, followed by an addition of 19.2 g of ST30, 7.7 g of 1034A, and then the mixture was stirred overnight. Then 24.0 g of PM and 31.6 g of EB9113 were added to the mixture and stirred for 1 hour, followed by addition of 0.9 g of IRGA 1173, 1.5 g of IRGA 184 and 0.1 g of EB1360. The resulting mixture was stirred at room temperature for 1 hour and then filtered through a 1.2 micrometre filter.

Comparative Example 2A mixture of 3.0 g of SR238 and 1.9 g of A1100 was stirred at room temperature in a plastic beaker for 2 hours. Then 5.6 g of PM, 20.9 g of IPA, and 0.3 g of Acrylic Acid were added to the mixture and stirred for another 0.5 hour, followed by an addition of 16.5 g of ST30, 6.8 g of 1034A and then the mixture was stirred for 1 hour. 0.8 g of IRGA 1173, 1.4 g of IRGA 184 and 0.1 g EB1360 were then added to the mixture and the mixture was stirred overnight. Then 42.7 g of EB9113 was added to the mixture and the resulting mixture was stirred at room temperature for 1 hour and then filtered through a 1.2 micrometre filter.

### Coating Application Process

### Dip Process

Coating compositions were applied to polymethylmethacrylate (PMMA) coupons (in size of 32 mm×28 mm×3 mm) by dip-coating at a withdrawal rate of 5.0 inches per minute (ipm) at room temperature.

### Brush Process

Coating compositions were brushed on PMMA coupons (in size of 32 mm×28 mm×3 mm) using a Hera Ceram brush from Kulzer GmbH, Germany. The brush was dipped into the coating composition for about 2 to 5 seconds. Any excess coating was removed by gently brushing against the wall of the coating container. The coating was then brushed onto a clean PMMA coupon in a single horizontal motion with a slight overlap between successive brush strokes in order to cover the entire substrate (e.g., dental appliance) surface. The coated substrates were allowed to dry in ambient air for about 5 minutes before cure. The coated substrate was cured by HiLite Power 3D™ unit from Kulzer GmbH, Germany by placing the coated substrate into the chamber of the curing unit, setting cure time of 15 minutes, and radiation curing the samples to provide a cured coating having a thickness of 3.0-6.0 micrometre.

### Physical Property of Liquid Coating and Performance of Cured Film

TABLE 2

| | | | | | Uncoated |
|---|---|---|---|---|---|
| | | | | | PMMA Coupon - |
| | Comparative | | | Comparative | Comparative |
| | Example 1 | Example 1 | Example 2 | Example 2 | Example A |
| Viscosity [cP @ | 6.0 | 5.5 | 6.6 | 5.5 | N/A |
| 25° C.] | | | | | |
| Application Process | Brush | Brush | Brush | Brush | N/A |
| FT [um] | 3.8 | 4.1 | 5.4 | 4.4 | N/A |
| % initial Adhesion | 100 | 100 | 100 | 100 | N/A |
| Erichsen Pencil | 10-11 | 10-11 | 10-11 | 10-11 | 6-7 |
| Hardness (N) | | | | | |
| 70° C. Stability | 6 | 10+ | less than 1 | less than 1 | N/A |
| (week) | | | | | |
| 40° C. Stability | 10+ | 10+ | 1.5 | 1.5 | N/A |
| (month) | | | | | |
| 7 days Discoloration | 0.09 | 0.11 | 0.37 | 1.01 | 0.30 |
| (ΔE*ab) | | | | | |

### Test

### Viscosity

The viscosity of the liquid coating composition was measured using a Brookfield viscometer Model DV2T LV (Ametek Brookfield, Boston, MA) at 25 degree C. and reported in centipoise (cP) units. 16 mL of liquid coating was placed in the ULA-31EY chamber of the viscometer and an ULA-E spindle was used for the viscosity measurement. The viscosity was recorded after the spindle was rotated for 5 min in the range of torque reading between 45% and 95%. The test results are shown in Table 2.

### Coated Film Thickness

The film thickness of the cured coating composition was measured with a Filmetrics F20 Thin Film Analyzer (Filmetrics, San Diego, CA) and reported in micrometer units. The test results are shown in Table 2.

### Adhesion

The adhesion was tested using 3M Brand SCOTCH 600 pressure-sensitive tape. The test was carried out as follows: 1) A 6×6 cross-hatch grid with approximately 2 mm grid spacing was made with a razor blade into the cured coating; 2) A rectangular piece of tape wide enough to fully cover the cross-hatched area but longer than the cross-hatched area was cut and the adhesive side was pressed down firmly over the cross-hatched area using a tongue depressor; 3) After a 90 seconds wait, the long end of the tape was grasped with one hand and pulled rapidly across the cross-hatched area at an angle of ˜180 degrees while firmly holding down the specimen with the other hand; 4) A check for the removal of the coating was made by examination of the coated substrate (e.g., dental appliance) using appropriate visual control; 5) Steps 2, 3 and 4 were repeated two additional times in the same cross-hatched area, each time using a new piece of tape; 6) The subject area was inspected under a microscope; 7) The actual count of unaffected areas was reported as percent adhesion. The test results are shown in Table 2.

### Erichsen Pencil Hardness

Pencil hardness of the coated specimen was evaluated using Erichsen pencil hardness tester, model 318S (Erichsen GmbH &Co, Germany). The spring tension was pre-set to 5 N using the slider on the Erichsen pencil. While holding the pencil upright, the stylus was placed on the specimen surface, and a 5 to 10 mm long scratch was made on the surface at a rate of approximately 10 mm/s. The surface was inspected by naked eye to observe the resulting scratch. If the scratch was not visible, the spring tension was increased in 1 N increments and the process repeated until the scratch was just visible to the naked eye. The corresponding spring tension value was recorded as Erichsen Pencil Hardness in Newton(N). The test results are shown in Table 2.

### 70 Degree C. Stability Testing

100 g of liquid coating was added to 125 mL amber HDPE bottle (VWR, Randor, PA) without cap. The HDPE bottle was placed into a stainless steel container (Model: STB-250) and then sealed with a cap (model: ENDCAP-205) using a clamp (Both container and cap are from Eagle Stainless, Warminster, PA). The stainless container was placed in a conventional oven. The oven was heated to 70 degree C. and the temperature of the oven was maintained at 70 degree C. for the entire stability testing. The stainless steel container was moved out from the oven after one week of the testing. After cooled down to room temperature, the stainless steel container was opened and about 20 g of the coating was removed from the HDPE bottle for a viscosity measurement. After completion of the viscosity measurement, the liquid coating was poured back to the HDPE bottle and the stainless steel container was sealed and placed in the 70 degree C. oven again for another week of the stability study. The same procedure was repeated weekly up to 8 weeks.

### 40 Degree C. Stability Testing

100 g of liquid coating was added to 125 mL amber HDPE bottle (VWR, Randor, PA) and sealed with cap. The HDPE bottle was placed in a conventional oven. The oven was heated to 40 degree C. and the temperature of the oven was maintained at 40 degree C. for the entire stability testing. The HDPE bottle was moved out from the oven after one month of the testing. After cooled down to room temperature, the HDPE bottle was opened and about 20 g of the coating was removed from the HDPE bottle for a viscosity measurement. After completion of the viscosity measurement, the liquid coating was poured back to the HDPE bottle, sealed with cap and placed in the 40 degree C. oven again for another month of the stability study. The same procedure was repeated monthly up to 6 months.

### 7 Days Discoloration (ΔE*Ab)

The discoloration of coated specimens was evaluated using instant coffee (UCC Ueshima Coffee Co., Ltd., Japan). A piece of specimen in the size of 20 mm×70 mm×1-1.5 mm was prepared. 6 g of the instant coffee was dissolved in 140 ml of hot water. The coffee solution was naturally cooled to 37 degree C. The piece of specimen was soaked and maintained in the coffee solution at 37 degree C. for 7 days. The specimen was rinsed in a running water and dried. The dried specimen was evaluated for the color differences (ΔE*ab) before and after the soaking using an integrating sphere spectrophotometer (Sakata Inx Eng. Co., Ltd., Japan). The test results are shown in Table 2.

Some examples of the present disclosure can exhibit excellent adhesion. For example, the adhesion of Examples 1 and 2 Comparative Example 1 and 2 is 100%.

Some examples of the present disclosure can exhibit excellent hardness. For example, the Erichsen Pencil Hardness (N) of Examples 1 and 2 Comparative Example 1 and 2 is 10-11 N.

In some examples of the present disclosure, multifunctional (meth)acrylate monomer/oligomer components are treated by silane (e.g., gamma-aminopropyl triethoxy silane). When a multifunctional (meth)acrylate monomer/oligomer component is treated by silane, it is believed that the composition becomes less stable. For example, when multifunctional (meth)acrylate monomer/oligomer components are treated by silane (e.g., gamma-aminopropyltriethoxysilane), the samples exhibit shorter 70 degree C. Stability (week) and/or 40 degree C. Stability (month).

When multifunctional (meth)acrylate monomer/oligomer components are not treated by silane (e.g., gamma-aminopropyltriethoxysilane), the samples exhibit longer 70 degree C. Stability (week) and/or 40 degree C. Stability (month). For example, the 70 degree C. stability (week) of Comparative Example 1 that is not treated by silane is 6+ weeks, the 70 degree C. Stability (week) of Example 1 that is not treated by silane is 10+ weeks, while the 70 degree C. Stability (week) of Example 2 and Comparative Example 2 that is treated by silane is less than 1 week. The 40 degree C. stability (month) of Comparative Example 1 and Example 1 that are not treated by silane is 10+ months, while the 40 degree C. Stability (month) of Example 2 and Comparative Example 2 that is treated by silane is 1.5 months.

In some examples of the present disclosure, the coating composition can reduce and/or prevent discoloration. For example, the 7 days discoloration (ΔE*ab) of Comparative Example 1 is 0.09 and the 7 days discoloration (ΔE*ab) of Example 1 is 0.11, while the 7 days discoloration (ΔE*ab) of an uncoated PMMA Coupon-Comparative Example A is 0.3.

### Plaque Adhesion to Coated Material

The anti-plaque property of the cured coating of the coating composition was evaluated by the following method. The coated PMMA coupons (in size of length: 75 mm, width: 25 mm, height: 1 to 1.5 mm: tolerance −0.1 mm) were prepared by the above mentioned Brush process as Samples. Uncoated PMMA coupons (in size of length: 75 mm, width: 25 mm, height: 1 to 1.5 mm: tolerance −0.1 mm) were prepared as reference. After sterilization (80% ethanol followed by rinsing in sterile distilled water) the samples were used immediately for testing. Dynamic in vitro biofilm testing to the samples was conducted using sterile flow chambers. For sealing of the flow chambers a glass slide was put under the PMMA coupons. Cross flow of the suspension of a mixed bacterial suspension was across the sample. The mixed bacterial suspension consisted of a mixed culture of five typical dental microorganisms (Streptococcus mutans, Streptococcus sanguinis, Actinomyces viscosus, Fusobacterium nucleatum, Veillonella parvula (bacterial concentration of each strain: appr. 2×106 bacteria per ml, total bacteria concentration: appr. 1×107 per ml)). Cultivation conditions were as follows: 37 degree C., pH=7.2, microaerophilic to anoxic conditions, BM medium supplemented with 0.5 g/L saccharose, volume flow rate of bacterial suspension of about 0.3 ml per min, laminar flow, incubation time: 23 hours, continuous cultivation under sterile conditions (chemostat).

Quantitative analysis of adherent bacteria was performed by confocal laser scanning microscopy (CLSM: Zeiss LSM 710, exc.: 488 nm and 543 nm, two separate detector channels objective: LD Plan-Neofluar 20x/0,4 Korr M27) at three to five sites of the sample surface after 23 hours of incubation. The samples were fluorescent stained (LIVE/DEAD BacLigh Bacterial Viability Kit [Life Technologies, Thermo Fisher Scientific Inc.; living bacteria with Syto 9=green, dead bacteria with propidium iodide=red]) directly within the flow chamber. The test results are shown in Table 3.

TABLE 3

| | | Coverage related to |
|---|---|---|
| | Coating | uncoated PMMA (%) |
| Example | Example 1 | 18 |
| | Example 2 | 30 |
| Comparative | (non-coated PMMA = adhered | 100 |
| Example | surface was set as reference at | |
| | 100%) | |
| | Comparative Example 1 | 92 |
| | Comparative Example 2 | 191 |

In some examples of the present disclosure, the coating composition can reduce and/or prevent plaque adhesion/coverage. The plaque coverage in Example 1 is 18% and the plaque coverage in Example 2 is 30%, while the plaque coverage in Comparative Example 1 is 92%, the plaque coverage in Comparative Example 2 is 191% and the non-coated PMMA is 100%.

The shelf life of Comparative Example 1 is 6 weeks and the shelf life of Example 1 is more than 10 weeks, while the shelf life of G-coat™ is 1 week, the shelf life of nano coat Labo™ and Palaseal™ is more than 9 weeks, respectively.

### Water Contact Angle of Coated Material

The water contact angle of the cured coating of the coating composition was measured on an example dental appliance. The example dental appliance was prepared as follows: Palapress (Registered Trademark) vario (acrylic resin dental base, Kulzer, Germany) was mixed in a ratio of powder: liquid=10 g/7 ml. The mixture was poured into a stainless steel mold. The mold heated in a hot water was pressured at 2 atmospheres at 55 degree C. for 30 minutes to polymerize the mixture. The surface of the solidified example dental appliance was polished by wet sanding using #400-#4000 sandpapers until the surface roughness (Ra) becomes less than 0.05 micrometre to obtain a dental appliance with a smooth surface.

The liquid coating composition was applied to the surface of the example dental appliance by brushing. The coated composition was air dried for 5 minutes and then cured by light irradiation using HiLite power 3D™ (Kulzer, Germany) for 15 minutes. The water contact angle of the cured coating on the surface of the example dental appliance was measured using a water contact angle meter, DropMaster DMo-501™ (Kyowa Interface Science Co., Ltd., Japan).

G-coat™, nano coat Labo™ and Paraseal™ was applied to the example dental appliance in the same manner, respectively. The coated surface of the example dental appliance was covered by Lumirror™ (polyester film, Toray Industries, Inc., Japan). The coated surface was formed on the surface of the example dental appliance by light irradiation using Solidilite V™ (Shofu Inc., Japan) for 3 minutes. The example dental appliance without applying any coating (non-coated base material) was also tested in the same manner. The test results are shown in Table 4.

TABLE 4

| | | Water |
|---|---|---|
| | | contact angle |
| | Coating composition | (°) |
| Example | Example 1 (Brush) | 83.4 |
| Comparative | — | 74.2 |
| Example | (non-coated base material) | |
| | (Comparative Example B) | |
| | G-coat ™ | 66.3 |
| | nano coat labo ™ | 65.4 |
| | Palaseal ™ | 69.0 |
| | Comparative Example 1 | 80.1 |

Example 1 of the present disclosure can exhibit high water contact angle. For example, the water contact angle of Example 1 is 81 degrees or more, while the water contact angle of non-coated base material as Comparative Example B, G-coat™, nano coat Labo™ and Palaseal™ is 74.2 degrees or less, respectively.

Some examples of the present disclosure can exhibit both excellent stability and high water contact angle. For example, Comparative Example 1 shows the water contact angle of 80.1 degrees and the 70 degree C. Stability (week) of 6+ weeks; 40 degree C. Stability (month) of 10+ months. Example 1 shows the water contact angle of 83.4 degrees and the 70 degree C. Stability (week) of 10+ weeks; 40 degree C. Stability (month) of 10+ months.

Water contact angle is an indicator that shows the material's water repellency. When the water contact angle is higher, the material has a higher water repellency. It is believed that a coating composition with a higher water contact angle has a higher microbial resistance (i.e., higher antimicrobial effect).

### Toothbrush Abrasion Test

The wear resistance of the cured coating composition was evaluated using SURFCOM 130A (Tokyo Seimitsu Co., Ltd., Japan). The roughness of the surface of the coating composition cured on the surface of the example dental appliance was measured using SURFCOM 130A. A test piece of 20 mm×70 mm×1-1.5 mm was prepared and set to a sample holder of a 6-station abrasion tester (K906-01, Tokyo Giken, Inc., Japan). The test piece was soaked in an aqueous solution including toothpaste, White and White Ca™ (Lion Corporation, Japan), in a volume ratio of powder/solution=1/2. The test piece was then brushed by a toothbrush for denture at a stroke strength of 250 g at a speed of 160 rpm with a stroke length of 3 cm. The surface roughness of the coated material on the test piece after 10,000 cycles of the brushing was measured. The test results are shown in Table 5.

TABLE 5

| | | Surface | Surface |
|---|---|---|---|
| | | roughness | roughness |
| | | (Before | (after 10,000 |
| | | toothbrush | cycles of |
| | | abrasion test) | brushing) |
| | Coating composition | (μm) | (μm) |
| Example | Example 1 (Brush) | 0.051 | 0.064 |
| Comparative | — | 0.041 | 0.859 |
| Example | (non-coated base material) | | |
| | (Comparative Example B) | | |
| | G-coat ™ | 0.055 | 0.192 |
| | nano coat labo ™ | 0.049 | 0.135 |
| | Palaseal ™ | 0.049 | 0.282 |
| | Comparative Example 1 | 0.041 | 0.055 |

Some examples of the present disclosure can exhibit improved resistance against toothbrush abrasion as compared to the abrasion observed in the non-coated base material. For example, the surface roughness (after 10,000 cycles of brushing) of Example 1 and Comparative Example 1 is 0.064 μm or less, while the surface roughness of G-coat™ is 0.192 μm, the surface roughness of nano coat Labo™ is 0.135 μm, and the surface roughness of Palaseal™ is 0.282 μm.

### Color Resistance of Coated Material

(Coffee, 37 degree C., 4 weeks)

The discoloration of coated specimens was evaluated using instant coffee (UCC Ueshima Coffee Co., Ltd., Japan). A piece of specimen in the size of 20 mm×70 mm×1-1.5 mm was prepared. 6 g of the instant coffee was dissolved in 140 ml of hot water. The coffee solution was naturally cooled to 37 degree C. The piece of specimen was soaked and maintained in the coffee solution at 37 degree C. for 4 weeks. The specimen was rinsed in a running water and dried. The dried specimen was evaluated for the color differences (ΔE*ab) before and after the soaking using an integrating sphere spectrophotometer (Sakata Inx Eng. Co., Ltd., Japan). The test results are shown in Table 6.

TABLE 6

| | | color |
|---|---|---|
| | | difference after |
| | | 4 wk immersion |
| 0 | Coating composition | (ΔE*ab) |
| Example | Example 1 (Brush) | 0.38 |
| Comparative | — | 0.69 |
| Example | (non-coated base material) | |
| | (Comparative Example B) | |
| | G-coat ™ | 0.34 |
| | nano coat labo ™ | 0.57 |
| | Palaseal ™ | 0.45 |
| | Comparative Example 1 | 0.31 |

In some examples of the present disclosure, the coating composition can reduce and/or prevent discoloration. For example, the 4 weeks discoloration (ΔE*ab) of Comparative Example 1 is 0.31 ΔE*ab, the 4 weeks discoloration (ΔE*ab) of Example 1 is 0.38 ΔE*ab.

It should be evident that this disclosure is by way of example and that various changes can be made by adding, modifying or eliminating details without departing from the fair scope of the teaching contained in this disclosure. The disclosure is therefore not limited to particular details of this disclosure except to the extent that the following claims are necessarily so limited.

1. A coated dental appliance or natural tooth comprising:
a dental appliance or natural tooth; and
a coating composition affixed on at least one portion of the dental appliance or natural tooth, said coating composition comprising:
at least one trifunctional or higher multifunctional (meth)acrylate monomer;
an inorganic filler;
at least one free-radical photoinitiator; and
an organic solvent.

2. The coated dental appliance or natural tooth of claim 1, wherein the coating composition comprises at least one multifunctional (meth)acrylate oligomer.

3. The coated dental appliance or natural tooth of claim 2, wherein the multifunctional (meth)acrylate oligomer is a hexafunctional aromatic urethane (meth)acrylate oligomer.

4. The coated dental appliance or natural tooth of claim 1, wherein the inorganic filler is amorphous nanosilica.

5. The coated dental appliance or natural tooth of claim 1, wherein the trifunctional or higher functionality (meth)acrylate monomer is trimethylolpropane triacrylate.

6. The coated dental appliance or natural tooth of claim 1, wherein the coating composition comprises difunctional (meth)acrylate monomers or oligomers.

7. The coated dental appliance or natural tooth of claim 1, wherein the coating composition comprises 1 to 30 parts by weight of the amorphous nanosilica in the coating composition.

8. The coated dental appliance or natural tooth of claim 1, wherein the coating composition comprises 5 to 25 parts by weight of amorphous nanosilica in the coating composition.

9. The coated dental appliance or natural tooth of claim 1, wherein the coating composition comprises at least 10 weight % of an organic non-reactive solvent.

10. A method of producing the coated dental appliance or natural tooth according to claim 1 comprising:
applying the coating composition on a surface of the dental appliance or natural tooth;
drying the applied coating composition on the surface of the dental appliance or natural tooth; and
curing the applied coating composition on the surface of the dental appliance or natural tooth.

11. The method of producing the coated dental appliance or natural tooth of claim 10, wherein the dental appliance is a full denture, a partial denture, a denture base, an artificial tooth, a dental prostheses, a splint, a dental aligner, a crown or a bridge.

12. The method of producing the coated dental appliance or natural tooth of claim 10, wherein the coating composition is applied by brushing the coating composition onto the dental appliance or natural tooth.

13. The method of producing the coated dental appliance or natural tooth of claim 10 wherein the coating composition is applied by dipping the dental appliance into the coating composition.

14. The method of producing the coated dental appliance or natural tooth of claim 10, wherein the coating composition is applied by spraying the coating composition onto the dental appliance or natural tooth.

15. The method of producing the coated dental appliance or natural tooth of claim 10, wherein the drying comprises air-drying at room temperature for at least 2 minutes.

16. The method of producing the coated dental appliance or natural tooth of claim 10, wherein the coating composition is cured by light in a wavelength range of 300 nm to 500 nm.

17. The method of producing the coated dental appliance or natural tooth of claim 10, wherein the coating composition is applied to the surface of the dental appliance or the natural tooth without any adhesion promoter or bonding agent being part of the coating composition or applied before the coating composition.

18. A coated dental appliance or natural tooth manufactured by the method according to claim 10.

19. The coated dental appliance or natural tooth of claim 18, wherein the coated dental appliance or natural tooth reduces plaque adhesion on its surface when compared to a corresponding uncoated dental appliance or natural tooth by more than 50% after 23 hours of incubation.

20. The coated dental appliance or natural tooth of claim 19, wherein the coated dental appliance or natural tooth reduces plaque adhesion on its surface when compared to a corresponding uncoated dental appliance or natural tooth by more than 70% after 23 hours of incubation.

21. The coated dental appliance or natural tooth of claim 19, wherein the coated dental appliance or natural tooth reduces plaque adhesion on its surface when compared to a corresponding uncoated dental appliance or natural tooth by more than 80% after 23 hours of incubation.

22. The coated dental appliance or natural tooth of claim 18, wherein the coated dental appliance or natural tooth has a surface hardness of at least 9 N as measured by Erichsen Pencil Hardness test.

23. The coated dental appliance or natural tooth of claim 18, wherein the cured coating composition on the dental appliance or natural tooth has a layer thickness of 1 to 100 micrometre.

24. The coated dental appliance or natural tooth of claim 18, wherein the cured coating composition on the dental appliance or natural tooth has a layer thickness of 3 to 60 micrometre.

25. The coated dental appliance or natural tooth of claim 18, wherein the cured coating composition is optically clear and colorless.

26. A coating composition for a dental appliance or natural tooth, comprising:
a (meth)acrylate component comprising a trifunctional or higher multifunctional (meth)acrylate monomer;
an inorganic filler;
a photoinitiator; and
an organic solvent.

27. The coating composition of claim 26, wherein a water contact angle of a cured coating of the coating composition is 75 degrees or more and less than 90 degrees.

28. The coating composition of claim 26, wherein a water contact angle of a cured coating of the coating composition is 81 degrees or more.

29. The coating composition of claim 26, wherein surface roughness of a cured coating of the coating composition after 10,000 cycles of tooth brushing is 0.100 μm or less.

30. The coating composition of claim 26, wherein coffee discoloration resistance of a cured coating of the coating composition is 3.0 ΔE*ab or less.

31. The coating composition of claim 26 further comprising 2-hydroxy-2-methyl-1-phenyl-propan-1-one and 1-hydroxy-cyclohexyl-phenyl-ketone.

32. The coating composition of claim 26, wherein the (meth)acrylate component further comprises a multifunctional urethane (meth)acrylate oligomer, and the (meth)acrylate component comprises 80 weight % or more and 100 weight % or less of the trifunctional or higher multifunctional (meth)acrylate monomer and the multifunctional urethane (meth)acrylate oligomer with respect to a total weight of the (meth)acrylate component.
